(19) United States
(12) Patent Application Publication
(10) Pub. No.: US 2004/0029171 A1
(43) Pub. Date: Feb. 12, 2004

(54) Method for carrying out a saliva analysis

(76) Inventors: Ingo Wagner, Steinebach (DE); Ingo Haeberlein, Weilheim (DE); Marc Peuker, Schondorf (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/332,844

(22) PCT Filed: Jul. 16, 2001
(86) PCT No.: PCT/EP01/07778

(30) Foreign Application Priority Data
Jul. 14, 2000 (DE) 10034647.2

(51) Int. Cl. G01N 33/53

(52) U.S. Cl. 435/7.1

(57) ABSTRACT

The invention relates to a method for analysis and to a device for carrying out the method. The method comprises the following steps: a) removing an applicator from a device comprising a pocket for receiving an applicator and at least one first compartment for receiving an indicator substance, said compartment having means for being connected to the pocket in the form of a connecting area to be opened selectively; b) wetting the applicator with a substance, for example saliva; c) introducing the applicator into the device; d) activating the device by exerting pressure on a compartment or by inserting the applicator into the compartment.

The invention relates to a method for analysis and a device for carrying out a method for analysis, in particular of saliva, which is suitable in particular for the detection of caries-causing and/or parodontitis-causing bacteria, which can be found, for example, in saliva and/or gum pocket fluid.

In the investigation of the causes of caries and parodontitis, individual bacteria and substances, inter alia, were identified which are possible as causative agents of these diseases. As soon as microorganisms of this type are found in the mouth, the destruction of the dental hard and soft tissue begins.

It would be desirable to have a method and a device with which the individual caries and/or parodontitis risk can be determined in a simple manner, in order to be able to initiate a treatment early enough.

Consequently, it is an object of the present invention to make available a simple method suitable for the above application and a suitable device.

This object is achieved by means of a method and a device such as are described in the claims.

The following devices are known from the prior art.

In EP-A-0895943, a device for the storage and application of a flowable substance is described. The brush found in the device serves for the application of the substance to a treatment site.

In WO-96/03326, a container for single use is described, for example, which has hollows for the storage of a medicament and of an applicator. Both hollows are protected from contamination by means of a peelable covering film. In one embodiment, the medicament is transferred to the hollow containing the applicator by pressing on the hollow containing the medicament in order to wet the applicator.

U.S. Pat. No. 3,835,834 relates to a transport container for culture media. It is mentioned that for use an absorptive applicator is wetted with the culture medium after opening the sterile pack and removing the applicator this is taken back to the transport container again. The transport container is then closed and a nutrient solution which is intended to prevent dying of the culture medium during transport is applied to the absorptive, wetted applicator.

According to the invention, it has been found that modifications of these devices are suitable for carrying out a method for analysis.

The device employable according to the invention comprises at least one first chamber, which is formed by a first and a second film. Into this chamber, one component of an indicator is at least partly introduced. The device further comprises a pocket separate from the chamber, which for its part has a prechamber or functions as such, the separation between the pocket and the first chamber having a passage region which is to be opened selectively.

According to the invention, the method comprises the following steps:

a) taking out an applicator from the device which contains an applicator, the applicator being situated either in an outwardly open pocket or in a closed container which, after opening the device and the container and taking out the applicator, forms an outwardly open pocket,

b) bringing the applicator into contact with a substance, for example saliva, in particular gum pocket fluid,

c) inserting the applicator into the device, preferably into the pocket from which the applicator was taken out or into a separate opening, for example into the pocket formed after opening the device,

d) optionally bringing the contaminated applicator from step b) into contact with a nutrient solution which is situated in a second chamber and which, by exertion of pressure, can be applied to the applicator from this chamber via a passage region to be opened selectively,

e) activating the device by exertion of pressure on a first chamber containing the indicator substance or by pushing the applicator into this chamber, the applicator being brought into contact with the indicator substance,

f) optionally taking out the applicator after a period of time which is adequate in order to generate a detectable signal,

g) optionally bringing the applicator into contact with a stop reagent in order to end the generation of a detectable signal after a period of time, the stop reagent preferably being situated in a third chamber,

h) if appropriate comparing the detectable signal with a reference signal, for example a color chart, which is preferably applied to the device,

i) optionally carrying out a positive control to check the functionality of the test method, in particular if no signal or only a poorly detectable signal is present.

Applicator is to be understood as meaning any manually manipulatable article which is suitable to be brought into contact with another substance.

The applicator is preferably designed to be paintbrush- or swab-like. An application instrument having a spherical, brush or paintbrush hair-carrying tip has proven utilizable in this context. In particular, cotton swabs and sponges have proven particularly favorably utilizable. If appropriate, the cotton swab has absorptive areas at both ends. It is possible to apply to the second absorptive area, for example, substances which are necessary for carrying out a positive control. Furthermore, pipettes or spatulas can be used as an application instrument. Optionally, the applicator also has a tip which allows sample substance to be taken out, for example, through the skin.

Furthermore, when using the applicator it is convenient if the pocket is sealed off outwardly by the application instrument.

This can be achieved, for example, by the applicator having a self-tapping thread on the shaft. The insertion and taking out of the applicator into or from the pocket is then carried out by rotating the applicator.

An embodiment of this type also makes possible a controlled mixing of the substances situated in the pocket or the prechamber by rotating the applicator, without it being possible for substance to escape through the opening of the pocket.

In this embodiment, it is convenient if the pocket has at least in the opening area a metal-containing foil, into which a thread can be cut.

Pocket is to be understood as meaning the design of a section of the device in the form of a container open to one side, which is suitable to receive an applicator.

The applicator can be situated in this pocket from the start and is taken back into it again after wetting with a sample substance.

However, it is also conceivable that the applicator is inserted into a sealed container, from which it is taken out after opening the device, preferably in sterile form.

It is advantageous in this case if, when opening the device to take out the applicator, a pocket having a prechamber is simultaneously opened, into which the applicator is inserted after wetting with the sample substance.

The term indicator substance includes all substances which are suitable for generating a detectable signal with another substance, preferably in visually perceptible form.

Suitable indicator substances include, for example, pH indicators (for example Bromophenol Blue, Congo Red, Bromocresol Green, Oregon Green derivatives, Rhodol derivatives), redox indicators (for example Methylene Blue, 5-cyano-2,3-ditolyltetrazolium chloride (CTC), 2-(4-iodophenyl)-3-(4-nitrophenyl)-5-phenyl-2H-tetrazolium chloride (INT), 8-dimethylamino-2,3-benzophenoxazine (Meldola's blue), 1-methoxyphenazine methosulfate (MPMS), 5-(3-carboxymethoxyphenyl)-2-(4,5-dimethylthiazolyl)-3-(4-sulfophenyl)tetrazolium (MTS), 3-(4,5-dimethylthiazol-2-yl)-2,5-diphenyltetrazolium bromide (MTT), 3,3′-(3,3′-dimethoxy-4,4′-biphenylene)-bis[2-(4-nitrophenyl-5-phenyl)]-2H-tetrazolium chloride (NBT), nitrotetrazolium violet (NTV), phenazine methosulfate (PMS), sodium 3′-[1-[(phenylamino)carbonyl]-3,4-tetrazolium]bis(4-methoxy-6-nitro)benzenesulfonic acid (XTT), phenazine ethosulfate (PES, WST-1), fluorescence indicators (for example Oregon Green 488 BAPTA, Calcium Green, Calcium Orange, Calcium Crimson), chemoluminescence indicators, vitality indicators (for example 5-bromo-2′-deoxyuridine), color indicators (for example p-nitroaniline derivatives, 2-naphthylamine derivatives, 7-amino-4-methylcoumarin derivatives, 7-amino-4-chloro-methylcoumarin derivatives, 6-aminoquinoline derivatives, rhodamine derivatives, 5,5′-dithiobis(2-nitrobenzoic acid), monobromobimane derivatives, tetramethylrhodamine derivatives, eosin derivatives, erythrosin derivatives, Texas Red derivatives, coumarin derivatives, pyridyloxazole derivatives, benzofurazan derivatives, naphthalene derivatives, didansylcysteine, dansylaziridine, pyrene derivatives, Coomassie Blue).

Moreover, the indicator substances can be bonded covalently to enzymes, proteins, glycoproteins, lipopolysaccharides, polysaccharides, polyclonal and monoclonal antibodies, DNA, RNA, cell organelles or microorganisms. In addition, the indicator substances can be bound to silicones, polyethers, alginates, polyethylenes, polypropylenes, poly(meth)acrylates, polyurethanes, polycarbonates, polysulfides, polyvinyl chlorides, polyvinylpyrrolidones, polyvinyl alcohols or rubber, gelatin, waxes, fats.

The term sample substance or marker compound includes all substances which are suitable for generating a detectable signal with the indicator substance and indicating mouth disorders and healing processes, preferably in visually perceptible form. The term marker compound also includes microorganisms.

In particular, substances having organic constituents are meant by marker compounds.

The marker compounds are preferably present in liquid form. However, the aggregate states solid and gaseous are also possible.

Preferably, the marker compounds are found in body fluids which are secreted by glands, such as saliva, perspiration, tears. However, all other body fluids and secretion products, such as gum pocket fluid, urine, blood, vaginal fluid and sperm, are also included.

The diagnosable mouth disorders also include caries, early onset parodontitis, prepubertal parodontitis, juvenile parodontitis, rapidly proceeding progressive parodontitis (RPP), adult parodontitis, refractory parodontitis, gingivitis, halitosis, infections with Candida albicans, Candida krusei, Candida glabrata, Candida lusitaniae, Candida dubliniensis, cancer, aphthous stomatitis, gingivostomatitis.

In the oral cavity, halitosis-causing bacteria can be found which release volatile sulfur compounds, such as mercaptans or hydrogen sulfide. In addition, dissimilatory sulfate-reducing bacteria are known whose hydrogen sulfide formation is correlated with the sulfate reduction.

By the use of the method according the invention, the formation rates of hydrogen sulfide and mercaptans, preferably methyl mercaptan, in gum pockets can be measured. Moreover, the bacterial enzyme activities, preferably methionine-γ-lyase, particularly preferably cysteine desulfhydrase, which catalyze the formation of the volatile sulfur compounds, can be measured as a gauge of the halitosis activity of gum pockets. Moreover, the presence of the bacteria responsible for the release, preferably Fusobacteria, Porphyromonas, Veillonella, Clostridium and Treponema, can be determined.

The various forms of parodontitis are causally connected with the infection by Actinobacillus Actinomycetemcomitans, Bacterioides forsythus, Campylobacter rectus, Capnocytophage ochracea, Capnocytophage gingivalis, Eikenella corrodens, Fusobacterium nucleatum, Porphyromonas asaccharolyticus, Porphyromonas gingivalis, Prevotella dentalis, Prevotella intermedia, Prevoltella nigrescens, Treponema denticola.

By the use of the method according the invention, the presence and amount of the bacteria can be determined. Suitable for this purpose are specific polyclonal antibodies and their subclasses or monoclonal antibodies which are directed against surface antigens of these bacteria, for example fimbriae, extracellular polysaccharides, adhesins.

By the use of the method according to the invention, it is furthermore possible to measure enzyme activities which give an indication of the presence and metabolic activity of a bacterium or of a group of the parodontitis-producing bacteria mentioned. The trypsin-like protease activity, preferably the dipeptidylpeptidase activity, particularly preferably Arg-gingipain activity and Lys-gingipain activity, is utilized diagnostically. For the determination of the Arg-gingipain activity, synthetic peptides can be employed which contain at least one Arg residue (in the P1 position) in addition to the detectable leaving group. For the determination of the Lys-gingipain activity, synthetic peptides can be employed which contain at least one one Lys residue (in the P1 position) in addition to the detectable leaving group. In addition to p-nitroaniline derivatives, for example Nα-benzoyl-DL-arginine-pnitroanilide or leucine-threonine-arginine-p-nitroanilide, 2-naphthylamine peptide derivatives, for example Nα-benzoyl-DL-arginine-β-naphthylamide, it is possible to employ 6-aminoquinoline peptide derivatives, rhodamine peptide derivatives and coumarin peptide derivatives, for example 7-amido-4-methylcoumarin, such as N-t-Boc-Val-Pro-Arg-7-amido-4-methylcoumarin and 7-amino-4-chloromethylcoumarin, such as N-t-Boc-Val-Pro-Arg-7-amido-4-chloromethylcoumarin, as detectable leaving groups.

By the use of the method according the invention, it is possible using polyclonal antibodies and their subclasses or monoclonal antibodies to diagnose the bacterial substances which lead to the induction of cytokines. Antibodies against lipopolysaccharides, lipoarabinomannan, peptidoglycans, teichoic acid derivatives, extracellular polysaccharides and lipid A are preferred.

By the use of the method according to the invention, it is also possible to diagnose the cytokine formation induced by parodontitis pathogens using polyclonal antibodies and their subclasses or monoclonal antibodies. Antibodies against the interleukins IL-1, IL-2, IL-3, IL-4, IL-5, IL-6, IL-7, IL-8, tumor necrosis factor TNFα, interferons α,β,γ, colony-forming factors M-CSF, growth factors EGF, TGFα and chemokines MCP can be employed.

By the use of the method according the invention, the destruction of parodontal tissue can be diagnosed by means of the enzyme activities of alkaline phosphatase, arylsulfatase, aspartate aminotransferase, β-glucuronidase, cathepsins (G,B,D), elastase, hyaluronidase, lactate dehydrogenase, lysozyme, matrix metalloproteinases (collagenases, gelatinases), tissue inhibitors metalloproteinases (TIMP), stromelysin, lactoferrin, tryptase and myeloperoxidase.

By the use of the method according to the invention, it is possible using polyclonal antibodies and their subclasses or monoclonal antibodies to diagnose the molecular markers for gingivitis. These include cytokines, for example interleukins IL-1, IL-2, IL-4, IL-6, TNFα and arachidonic acid derivatives, for example prostaglandin E₂.

Caries is causally connected with infection by Streptococcus salivarius salivarius, Streptococcus vestibularis, Streptococcus thermophilus, Streptococcus mutans, Streptococcus rattus, Streptococcus sobrinus, Streptococcus cricetus, Streptococcus downei, Streptococcus macacae, Streptococcus ferus, Streptococcus milleri, Streptococcus anginosus, Streptococcus constellatus, Streptococcus intermedius, Streptococcus mitis, Streptococcus oralis, Streptococcus sanguis, Streptococcus gordonii, Streptococcus parasanguis, Streptococcus crista, Streptococcus mitior, Lactobacillus acidophilus, Lactobacillus alimentarius, Lactobacillus brevis, Lactobacillus buchneri, Lactobacillus casei, Lactobacillus paracasei ss paracasei, Lactobacillus paracasei ss rhamnosus, Lactobacillus paracasei ss tolerans, Lactobacillus delbrueckii, Lactobacillus delbrueckii ss lactis, Lactobacillus delbrueckii ss delbrueckii, Lactobacillus delbrueckii ss bulgaricus, Lactobacillus endocarditis, Lactobacillus fermentum, Lactobacillus gasseri, Lactobacillus pseudoplantarum, Lactobacillus rhamnosus, Lactobacillus salivarius, Actinomyces israelii, Actinomyces odontolyticus, Actinomyces actinomycetemcomitans, Eikenella, Branhamella catarrhalis, Veillonella alcalescens, Veillonella parvula, Actinomyces naeslundii, Rothia dentocariosa.

By the use of the method according to the invention, it is possible using polyclonal antibodies and their subclasses or monoclonal antibodies which are directed against the various surface antigens of these bacteria, for example proteins, lipopolysaccharides, glycoproteins, fimbriae, extracellular polysaccharides, adhesins, lipoteichoic acid derivatives, glucan binding proteins, collagen binding proteins, to diagnose the presence and amount of the cariogenic bacteria.

By the use of the method according to the invention, extracellular enzyme activities of cariogenic bacteria can be diagnosed, for example proteases, preferably glucosyl transferases, glucanase, fructosyl transferase, fructanase.

By the use of the method according to the invention, metabolic products of cariogenic bacteria can be diagnosed, for example butyric acid, formic acid, preferably acetic acid, propionic acid, lactic acid, particularly preferably lactic acid. The acidification of the surrounding medium accompanying the release of acid can moreover be detected using pH indicators, for example using Bromophenol Blue, Congo Red, Bromocresol Blue, preferably Rhodol derivatives, particularly preferably Oregon Green derivatives.

As a result of the acidification of the pH in the surrounding medium, such as plaque, calcium ions are dissolved out of the dental hard substance. By the use of the carrier materials according to the invention and use of the method according to the invention, this process can be diagnosed using calcium indicators, for example Calcium Crimson, preferably Calcium Green, Calcium Orange, particularly preferably Calcium Oregon Green 488 BAPTA.

By the use of the method according to the invention, the increase or the decrease in the abovementioned marker compounds can be used as a measure of the course of the disorder and as a measure of the healing process.

Bringing in contact is to be understood as meaning the uptake of an amount of substance, using the applicator, which is sufficient to generate a detectable signal with the indicator substance.

The uptake can be carried out by physical and/or chemical processes, such as adsorption, absorption, adhesion, aspiration, wetting and/or diffusion.

The invention at the same time has the following advantages:

The method according to the invention allows a rapid, uncomplicated analysis of substance samples.

Due to the fact that all aids needed for the desired analysis are found in one device, a mix-up of substances needed for the analysis is excluded.

Furthermore, it is ensured by the device that the indicator substance is not contaminated before its use for analysis.

The method according to the invention in combination with the associated device makes possible the carrying out of an analysis of substances without time-consuming unscrewing and application of indicator solutions to samples taken out and can also be carried out by persons not previously trained technically and professionally.

A further advantage consists in the fact that all aids needed for the desired analysis are present in the device in the quantitative ratios needed. Thus erroneous dispensing of substances needed for the analysis is excluded.

The device employed in the method according to the invention is preferably intended for single use.

It can be advantageous if an area is situated on the device which makes possible a comparison of the generated signal with a reference signal.

Such a reference signal can be printed on, for example, in the form of a color scale, which is related to the signal generated by the indicator substance.

Preferably, in the area in which the analysis reaction takes place the device has a transparent window which makes possible the observation of the analysis reaction.

Optionally, this window is only visible after stripping off a protective film, preferably when opening the device.

The device according to the invention optionally has further chambers, preferably a second and a third chamber, which likewise in each case are connected with the pocket or another chamber via an area to be opened selectively. Into these further chambers, additional substances which are optionally necessary for the method beside the actual indicator substance can be introduced.

According to need, these additional substances can be transferred through the area to be opened selectively into another chamber or into the pocket by exertion of pressure on the respective chamber.

An embodiment of this type makes possible the use of the device for a large number of indicator substances, which, although on their own stable and storable per se, for use in the analysis additionally need activation by addition of other substances.

Such substances optionally additionally necessary for the actual indicator substance include buffer substances, such as phosphates, for example sodium phosphate, sodium hydrogenphosphate, sodium dihydrogenphosphate, potassium phosphate, potassium hydrogenphosphate, potassium dihydrogenphosphate, pyrophosphate, carbonates, for example sodium carbonate, potassium carbonate, sodium hydrogencarbonate, potassium hydrogencarbonate, acetic acid/acetate, citric acid/citrate, diethylbarbituric acid, tris(hydroxymethyl)aminomethane (TRIS), glycine, glycylglycine, N-(2-acetamido)-2-aminoethanesulfonic acid (ACES), N-(2-acetamido)iminodiacetate (ADA), N,N-bis(2-hydroxyethyl)-2-aminoethanesulfonic acid (BES), N,N-bis(2-hydroxyethyl)glycine (BICINE), 2,2-bis(hydroxyethyl)iminotris(hydroxymethyl)methane (BIS-TRIS), 2-(cyclohexylamino)ethanesulfonic acid (CHES), 2-[4-(2-hydroxyethyl-1-piperazine)]ethanesulfonic acid (HEPES), 3-[4-(2-hydroxyethyl-1-piperazinyl)]propanesulphonic acid (HEPPS), 2-morpholinoethanesulfonic acid (MES), 3-morpholinopropanesulfonic acid (MOPS), piperazine-1,4-bis(2-ethanesulfonic acid) (PIPES), N-[tris(hydroxymethyl)-methyl]-2-aminoethanesulfonic acid (TES), N-[tris-(hydroxymethyl)methyl]glycine (TRICINE), acids, such as sulfuric acid, sulfurous acid, phosphoric acid, hydrochloric acid, acetic acid, nitric acid, bases, such as sodium hydroxide, potassium hydroxide, lithium hydroxide, ammonia, calcium hydroxide, magnesium oxide, solvents, such as water, methanol, ethanol, isopropanol, propanol, glycerol, dimethyl sulfoxide, tetrahydrofuran, acetone, butanone, cyclohexane, toluene, methylene chloride, chloroform, alkanes, ethyl acetate, salts, such as magnesium chloride, magnesium sulfate, magnesium nitrate, calcium chloride, calcium sulfate, calcium nitrate, iron(III) chloride, iron(II) chloride, ammonium sulfate, sodium chloride, potassium chloride, sodium phosphates, potassium phosphates, coenzymes, vitamins, substrates for enzymes, such as sugar phosphates, lactic acid, pyruvate, acetic acid, acetate, propionic acid propionate, lipopolysaccharides, toxins, peptides, antibodies, such as polyclonal and monoclonal antibodies and their subclasses and/or enzymes, which can be subdivided into the following classes, the following list being by way of example and nonlimiting for the invention:

oxidoreductases and their subclasses, for example dehydrogenases, such as lactate dehydrogenase, oxidases, peroxidases, reductases, monooxygenases, dioxygenases;

transferases and their subclasses, for example C₁-transferases, glycosyl transferases, such as glucosoyl transferases, fructosyl transferases, aminotransferases, phosphotransferases;

hydrolases and their subclasses, for example esterases, glycosidases, such as glucanase, fructanase, peptidases, for example dipeptidylpeptidases, Arg-gingipain, Lys-gingipain, collagenases, gelatinases, cathepsins, elastase, amidases;

lyases and their subclasses, for example C—C lyases, C—O lyases, C—N lyases, C—S lyases;

isomerases and their subclasses, for example epimerases, cis-trans isomerases, intramolecular transferases;

ligases and their subclasses, for example C—C ligases, C—O ligases, C—N ligases, C—S ligases.

Over 2000 different enzymes are known today. For their classification, a system has been developed which takes into consideration action and substrate specificity. It results from this that specific substrates and/or coenzymes (for example NAD(P), NAD(P)H, FAD, FMN, lipoamide, ubiquinone, heme, ATP, ADP, AMP, GTP, GDP, GMP, UTP, UDP, UMP, CTP, CDP, CMP, coenzyme A, thiamine diphosphate, pyridoxal phosphate, biotin and/or tetrahydrofolate) belong to each enzyme.

These specific substrates and/or coenzymes are customarily present if, for example, one or more enzymes serve as a marker substance. Conversely, it is of course-true that specific enzymes can be used as necessary substances if specific substrates, for example sugar phosphates, lactic acid/lactate, pyruvate, acetic acid/acetate, propionic acid/propionate, formic acid/formate, peptides, synthetic peptides serve as sample substances or marker substances.

The substances necessary for the analysis situated in the device can be present in liquid or powder form. Preferably, they are present in a form which allows the bringing into contact of the indicator substances and/or stop reagents with the sample substance, which is situated on the applicator by the passage area or the passage areas to be opened selectively. Preferably, the reagents employed are present in dissolved form.

If appropriate, it is necessary to fix or to stabilize the sample substance to/on the applicator. This can be carried out by rebuffering, pH change, alteration of the ionic strength, addition of inhibitors against enzymatic degradation, stabilizers and/or fixatives.

The substances needed are situated either in a further chamber, which are connectable via a passage area to be opened selectively to the area in which the applicator is situated, or in a sealed container which is situated in a chamber and which breaks open on activating the device.

If appropriate, the device has an outer layer area and an inner layer area different therefrom, which forms the chambers. The outer layer area can be designed to be peelable, so that the outer layer area can be at least partly peeled off.

The use of differently constituted films for the outer layer area on the one hand and the films forming the chamber on the other hand allows safe storage of the substances before the device is put into use.

Moreover, the substances to be mixed remain better protected from external influences in the stored state, such as incidence of light or alternatively unwanted pressure stress.

If one of the films forming the chamber is transparent, after stripping off the peelable outer layer area the reaction proceeding between substance to be analysed and indicator substance and/or the emptying of the chamber into the pocket can be monitored, for example, visually.

Depending on indicator substance contained in the device, the inner layer of the film which comes into contact with the indicator substance can be adapted to the properties of the substance. This makes possible the use of the device for storage for a large number of indicator substances which are different in their reactivity, without the method for the production of the device having to be changed round altogether.

Optionally, in one chamber of the device there is additionally situated a separate compartment, preferably in the form of a pad, for the absorption of a substance, such as the actual indicator substance, an activating substance for the indicator substance or a substance which is necessary for the reaction between substance to be analyzed and actual indicator substance.

Such substances are, for example, buffer substances, acid, bases or solvents, as have already been described above.

The presence of a further compartment of this type prevents this substance from coming into contact in the stored state immediately either with the passage area to be opened selectively or with the films forming the chamber.

By this means, damage to the device, in particular to the films forming the device, is made difficult. This makes possible an increase in the storage time of the device containing the substance.

In this context, the use of materials severely adversely affecting or preventing the diffusion of volatile substances has proven advantageous for the production of the container.

The first and second films are optionally multilayer films. They have an optionally at least partly peelable outer layer area and an inner layer area.

The films preferably used are those which have an adequate diffusion tightness.

Depending on the condition of the substance to be stored, the films should also be resistant to aggressive, for example corrosive, substances and/or substances having solvent properties.

The film constituents can be chosen from plastic films, metal foils and ceramic films.

Plastics possible for the production of films are, for example: PE, PP, PTFE, PET, PA, PBT, PVC, EVA.

Metals possible for the production of foils are, for example: Al, Sn, Au, Ag, Fe, Cu.

Ceramic films are to be understood as meaning films which have, for example, an SiOx-containing layer.

The film construction-is fundamentally arbitrary and adapts to the composition of the substances to be stored.

A film construction having the sequence from outside to inside PET, Al, PET, PE or PP, Al, PET, PE, optionally also without PET film as the middle film, has proven advantageous.

Furthermore, adhesive additives can be situated between the films.

Possible adhesive additives are: laminating adhesives or extrusion lamination media.

The optionally present peelable outer layer area of the first film is preferably opaque.

The inner layer area of the first film is preferably transparent and optionally more flexible than the outer layer area.

The sequence from outside to inside PET, Al or PP, Al has proven advantageous for the outer layer area.

The separation between the chamber or chambers and the pocket is designed with respect to the distance and with respect to the firmness of the adhesion such that they can form a theoretical breakage site.

The passage area is constituted such that in the storage state it forms a tight seal, both to the pocket and to the optionally present second and/or third chamber.

Such a theoretical breakage point can be achieved, for example, by heat sealing or sticking, in the case of heat sealing a different energy input in comparison with the other sealing areas, preferably a lower energy input, taking place. This can be controlled by means of the temperature, pressure and/or holding time.

Another possibility consists in introducing foreign particles reducing the adhesion, such as peel film stampings or hot-melt adhesive spots, into the area of the theoretical breakage point between the first and the second films. In this case, tight-sealing films are preferably employed as the upper and lower films.

The chamber or the chambers are preferably designed to be round (circular or oval) but optionally also angular (square, rectangular or triangular).

According to their purpose, the chambers have specific volumes, so that in the case of use as directed and a plurality of components to be mixed homogeneous mixing can be carried out. The chamber adjacent to the pocket in this case preferably has a volume which is suitable for receiving the total amount of substance to be mixed or mixed substance.

The chamber volume can be in the range from a few tenths of milliliters, for example 0.01 ml to approximately 50 ml, a range from 0.01 to 10 ml is preferred, particularly preferably a range from 0.01 to 5 ml.

A chamber has, for example, a diameter of 5 to 20 mm, the application instrument a shaft diameter in the region of 2 mm.

The pocket is open to one side and optionally designed such that it can receive an application instrument even in the storage state. In the case of an appropriately small diameter of the pocket opening, the pocket can also itself serve outwardly, for example in the form of a cannula, as an application device.

The device comprises a container which includes a first film, for example a covering film, and a second film, for example a thermoformed film, such as is used in conventional blister packs.

With the exception of the areas forming the chambers, the films are preferably connected to one another flat.

The connection of the first to the second film can be carried out, for example, by heat sealing, cold sealing, sticking together and/or ultrasonic welding using sonotrodes.

A multilayer construction of the first and second film can be achieved by laminating, calendering, bonding various monofilm layers, optionally also by vapor deposition, for example with metals.

In order to protect the applicable substances incorporated into the device, for example from the incidence of light, the films are preferably designed such that they are connected to one another in an area surrounding the chamber by two sealing seams arranged at a distance from one another.

The container or containers can be produced by the same process which can be used for the production of the device. The container is in this case preferably produced by bonding, sticking together or sealing in the boundary area of plastic- or metal-containing films/foils so that a pad-shaped structure preferably results. The films/foils are connected in such a way that the container can be opened by the action of external pressure, the container being preferably intended to break open undirected on the action of pressure.

For the use of the device, the applicator is firstly taken out of the pocket and brought into contact with the substance to be analyzed, preferably dipped into the substance to be analyzed. The process of wetting the applicator customarily lasts for only a few seconds.

For the analyses of saliva, the applicator can be dipped either into a vessel filled with saliva or, in the simplest case, the saliva can be taken directly out of the mouth by bringing the applicator into contact with saliva in the mouth.

For the analysis of the gum pocket fluid, the applicator can be introduced, for example, into a gum pocket.

The wetted applicator is put back into an opening of the device, preferably into the pocket from which it was taken out.

The activation of the indicator substance then takes place, provided this is necessary. This can be carried out, for example, by exertion of pressure on a second chamber containing an activating substance. The exertion of pressure causes the activating substance to pass into the chamber in which the actual indicator substance is situated via the passage area to be opened selectively.

Alternatively, the activation can also be carried out by exertion of pressure on the chamber containing the indicator substance itself, if the activating substance is situated in a container which is optionally present in the chamber. This container, which is customarily pad-shaped, preferably breaks open undirected here.

The optionally activated indicator substance is then brought into contact with the wetted applicator. This can be achieved by exerting external pressure on the chamber containing the indicator substance, which leads to-opening of the passage area to be opened selectively to the pocket containing the applicator. The reaction between indicator substance and analysis substance then takes place in the pocket.

Alternatively, the applicator can also be pushed into the chamber containing the indicator substance through the passage area to be opened selectively. In this case, the reaction takes place in the chamber containing the indicator substance.

It is also possible that the applicator is pushed into an empty chamber through a first passage area to be opened selectively. The indicator substance, optionally together with an activating agent, is then transferred to the originally empty chamber via a further passage area to be opened selectively which is connected to this chamber. The reaction in this case takes place in the originally empty chamber.

After completion of the reaction, which customarily proceeds in a period of a few seconds up to a few minutes, the applicator is optionally taken out of the device. Depending on the indicator substance used, a detectable signal is obtained, preferably a visually recordable color change.

To end the formation of the detectable signal, a stop reagent can be introduced from a further pad.

Suitable stop reagents are, for example, acids, such as sulfuric acid, sulfurous acid, phosphoric acid, hydrochloric acid, acetic acid, nitric acid, citric acid, ascorbic acid, bases, such as sodium hydroxide, potassium hydroxide, lithium hydroxide, ammonia, calcium hydroxide, inhibitors, such as iodoacetic acid, monobromobimane derivatives, dansyl derivatives, hydrogen peroxide. If the substances necessary include one or more enzymes, the specific inhibitors known for the respective enzyme can in principle be used; for example, pyruvate or phosphate anions or pyrophosphate anions are inhibitors of lactate dehydrogenase.

A comparison with a reference signal, for example a color scale, which is preferably attached to the device, allows the assessment of the analysis carried out.

In a further preferred embodiment, the device comprises means for carrying out a positive control for the test process. Such a positive control is, for example, sensible when the applicator or the indicator situated on it does not change color or only changes color slightly after carrying out the analysis method. In such a case, the possibility in fact exists that the substance to be detected was not present in the sample investigated, or the possibility that the test as such was not functional.

For checking which of the possibilities exists, it is appropriate to subject the applicator to a positive control after carrying out the method.

A suitable positive control which can be used in a method for the detection of caries-causing and/or parodontitis-causing bacteria comprises, for example, a 1 to 10 mM lactate solution, optionally in a buffer of glycyl-glycine (about 50 mM) and sodium hydrogenphosphate (about 10 mM), at a pH of about 9.

For carrying out the positive control in this embodiment, the applicator is brought into contact with lactate after carrying out the actual method. Lactate in this case goes into solution and a color change of the indicator should or must occur if the test was functional.

The substances which are needed for the positive control are expediently situated in a separate container. The container can, for example, contain a non-woven fabric, and be attached, for example, to the back of the device. Also possible, however, is application of the container to the device in the form of a detachable blister or separate addition, for example in the form of a test strip.

Preferred exemplary embodiments for the design of the device are illustrated below with the aid of the drawings.

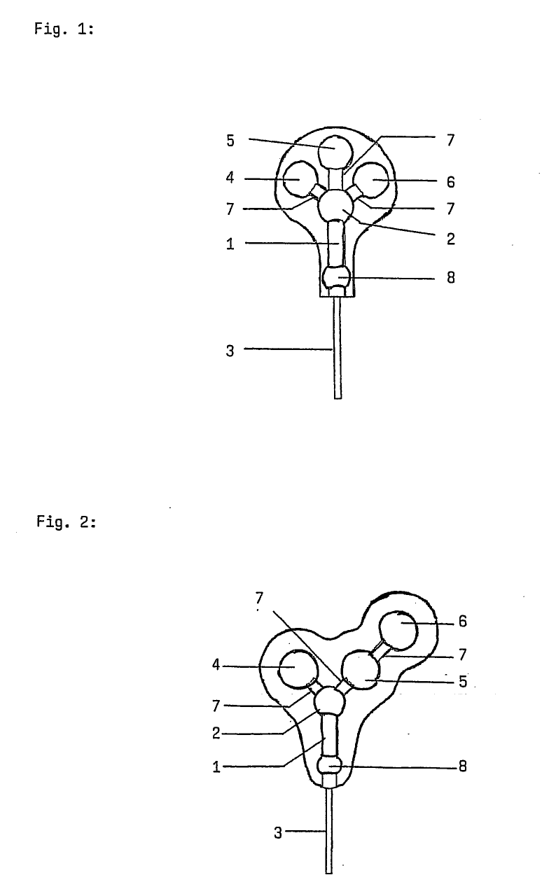

FIG. 1 shows an embodiment of the device having three chambers arranged in parallel.

FIG. 2 shows an embodiment of the device having three chambers, of which two are arranged serially.

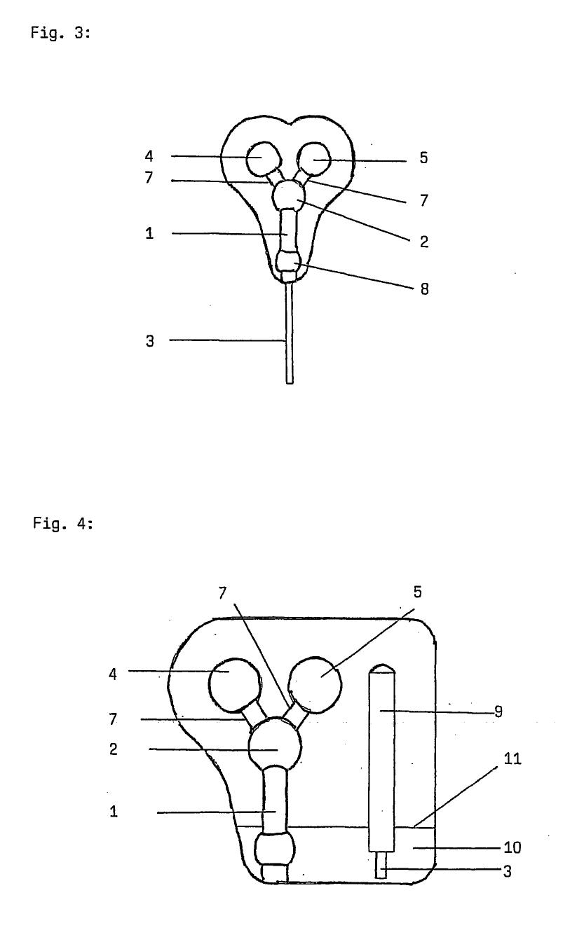

FIG. 3 shows an embodiment of the device having two chambers arranged in parallel.

FIG. 4 shows an embodiment of the device which makes possible sterile storage of the applicator.

FIG. 1 shows an embodiment of the device according to the invention having a pocket (1) which opens into a prechamber (2) and in which is situated an applicator (3), the shaft of the applicator projecting from the pocket (1). In the opening area of the pocket is optionally situated a stop aid (8) for the applicator. The prechamber (2) is optionally made of transparent material, so that the course of the analysis can be observed.

The device further comprises three chambers (4, 5, 6), which in each case open into the prechamber (2) of the pocket (1) via passage areas (7) to be opened selectively. This arrangement is referred to as a parallel arrangement of the chambers.

This arrangement is, for example, convenient when the applicator wetted with sample substance is additionally to be wetted with a nutrient solution, for example a sugar solution, which is situated in chamber (5), before bringing it into contact with an indicator solution which is situated in chamber (4). A stop reagent is in this case situated in chamber (6).

An optionally present color scale can be attached, for example, to the back of the device.

In FIG. 2, two of the three chambers present are arranged serially, i.e. in series. This arrangement is, for example, advantageous when the indicator substance, which is situated in chamber (5), firstly has to be activated. The activation solution is in this case preferably situated in chamber (6). Chamber (4) can contain either a nutrient solution or a stop reagent.

The device in FIG. 3 comprises only two chambers (4, 5) arranged in parallel. It is also possible that the device only has one chamber, in which the indicator solution is situated. Optionally, a further compartment can additionally be placed into this chamber, which compartment, for example, contains an activating solution for the indicator substance and, on activating, breaks out by exertion of pressure on the chamber.

If it is to be ensured that the applicator must be stored under sterile conditions, the device according to FIG. 4 has proven advantageous. In FIG. 4, the applicator (3) is situated in a container (9). This container and the opening of the pocket (1) with the prechamber (2) are tightly sealed in the starting state by the film (10), which can preferably be removed up to the fold (11). On opening the device, both the shaft of the applicator (3) and the opening of the pocket (1) is thus exposed.

The method according to the invention is suitable for the analysis of all substances which can wet the applicator contained in the device and which can react with the indicator substance contained in the device.

Preferably, the method is suitable for the rapid and simple analysis of body fluids, in particular of saliva and blood and the organic or biochemical substances contained therein.

In a particular embodiment, the method for the analysis of saliva is suitable for those substances contained therein which can be used for the determination of the individual caries and/or parodontitis risk.

In a further particular embodiment, the method for the analysis of saliva is suitable for those substances contained therein which can be used for the determination of the individual parodontitis risk.

For such a saliva test, the following indicator substances have proven advantageous: dyes which are detached from protease substrates by proteolytic activity. In particular, redox dyes, for example PMS, MTT, PES, CTT, MTS are suitable. However, all above-mentioned indicator substances are also suitable. Also included are peptide derivatives of p-nitroaniline, of 2-naphthylamine, of 7-amino-4-methylcoumarin, of 7-amino-4-chloromethylcoumarin, of 6-aminoquinoline, of rhodamine, N-benzoyl-DL-arginine-2-naphthylamide and benzoyl-DL-arginine-p-nitroanilide.

Likewise, the present invention relates to a kit for the site-specific determination of the individual caries and/or parodontitis risk by means of an impression compound, containing diagnostically active additives.

In the method on which this kit is based, the individual caries risk is firstly determined from saliva by means of the method described beforehand and in the case of a positive result an impression of hard tissue, which is rinsed with saliva, preferably of at least one region of the jaw, for example of the lower and/or upper jaw, is taken using a diagnostic impression compound.

Suitable diagnostic impression compounds are, for example, the compounds which are described in the application DE-199 26 728, in particular based on alginate, polyether, silicone or polyether silicone.

In this context, these are curable or film-forming carrier materials which contain diagnostically utilizable additives for site- and substance-specific intraoral diagnosis in a preferred amount of 0.0001 to 10% by weight. Suitable diagnostic additives according to this application are color indicators, antibodies, enzymes and all other substances with which the person skilled in the art familiar with the development of diagnostic test systems is familiar.

In the hardened impression material, impression compounds of this type show a perceptible signal, for example a coloration, on those sites at which an increased risk of caries is present. For this, selectively, metabolic products of the caries-promoting microorganisms are utilized as active species in the detection method used.

The invention is illustrated below with the aid of examples.

### EXAMPLE 1

Saliva Test 1

A device having a pocket for the applicator and a chamber is used. In the chamber are situated 200 μl of diagnosis solution, containing an amino compound, a coenzyme, a dehydrogenase and a redox indicator.

The applicator is taken out of the device, wetted with saliva in the mouth of the patient and put back into the pocket again. By pressing, the chamber is opened and the diagnosis liquid is transferred to the pocket through the passage area. By rotating the applicator, complete wetting of the tip of the applicator with diagnosis liquid is guaranteed. After approximately 3 min, the applicator can be taken out and the amount of the substance to be detected present in the saliva of the patient can be evaluated by the coloration of the tip of the applicator.

### EXAMPLE 2

Saliva Test 2

A device having a pocket for the applicator and two chambers arranged in parallel according to FIG. 3 is used. In the first chamber are situated 200 μl of diagnosis solution, containing an amino compound, a coenzyme, a dehydrogenase and a redox indicator. In the second chamber are situated 50 μl of stop reagent.

The applicator is taken out of the device, wetted with saliva in the mouth of the patient and put back into the pocket again. By pressing, the chamber containing the diagnosis liquid is opened and transferred to the pocket through the passage area. By rotating the applicator, complete wetting of the tip of the applicator with diagnosis liquid is guaranteed. After approximately 3 min, the second chamber containing the stop reagent is opened by pressing and the stop reagent is transferred to the pocket through the passage area. By rotating the applicator, complete wetting of the tip of the applicator with stop reagent is guaranteed.

The applicator can be taken out and the amount of the substance to be detected present in the saliva of the patient can be evaluated by the blue coloration of the tip of the applicator.

### EXAMPLE 3

Saliva Test 3

A device having a pocket for the applicator and two serially arranged chambers and one chamber arranged in parallel according to FIG. 2 is used.

In the upper serially arranged chamber are found 200 μl of diagnosis solution, containing an amino compound. In the lower serially arranged chamber is found the diagnosis powder, containing a coenzyme, a dehydrogenase, a redox indicator and a sugar-containing compound. In the chamber arranged in parallel are found 50 μl of acid as a stop reagent.

The applicator is taken out of the device, wetted with saliva in the mouth of the patient and put back into the pocket again. By pressing, the upper serially arranged chamber containing the buffer solution is opened and transferred through the passage area into the lower serially arranged chamber containing the diagnosis powder.

By mutual pressing on the two serially arranged chambers, the liquid is moved to and fro between the two chambers, whereby it is ensured that the diagnosis powder goes completely into solution. The diagnosis solution is then transferred through the passage area into the pocket. By rotating the applicator, complete wetting of the tip of the applicator with diagnosis liquid is guaranteed. After approximately 3 min, the chamber arranged in parallel containing the stop reagent is opened by pressing and the stop reagent is transferred through the passage area into the pocket of the applicator. By rotating the applicator, complete wetting of the tip of the applicator with stop reagent is guaranteed. The applicator can be taken out and the amount of the substance to be detected present in the saliva of the patient can be evaluated by the coloration of the tip of the applicator.

### EXAMPLE 4

Saliva Test 4

A device having a pocket for the applicator and two chambers arranged in parallel according to FIG. 3 is used. In the first chamber are situated 200 μl of diagnosis solution, containing an amino compound, a mixture of various salts, and a peptide-containing compound. In the second chamber are situated 50 μl of acid as a stop reagent.

The applicator is taken out of the device, wetted with saliva in the mouth of the patient and put back into the pocket again. By pressing, the chamber containing the diagnosis liquid is opened and transferred through the passage area into the pocket. By rotating the applicator, complete wetting of the tip of the applicator with diagnosis liquid is guaranteed. The formation of a yellow coloration can be observed through the sight window in the applicator pocket.

After approximately 3 min, the second chamber containing the stop reagent is opened by pressing and the stop reagent is transferred to the pocket through the passage area. By rotating the applicator, complete wetting of the tip of the applicator and thorough mixing of the solution with stop reagent is guaranteed. The reaction comes to a halt and the coloration can be evaluated using a color chart.

### EXAMPLE 5

Saliva Test 5

A device having a pocket for the applicator and a chamber is used. In the chamber are situated 200 μl of diagnosis solution, containing an amino compound, a mixture of various salts and a peptide-containing compound.

The applicator is taken out of the device, wetted with saliva in the mouth of the patient and put back into the pocket again. By pressing, the chamber is opened and the diagnosis liquid is transferred to the pocket through the passage area. By rotating the applicator, complete wetting of the tip of the applicator with diagnosis liquid is guaranteed.

After approximately 3 min, the applicator can be taken out and the amount of the substance to be analyzed present in the saliva of the patient can be evaluated by the coloration of the tip of the applicator.

List of Reference Symbols

1. pocket

2. prechamber

3. applicator

4. first chamber

5. second chamber

6. third chamber

7. passage area to be opened selectively

8. stop aid

9. container for receiving the applicator

10. pull-up film

11. opening fold

1. A method for analysis, comprising the following steps:
a) taking out an applicator from a device, comprising a pocket for receiving an applicator and at least one first chamber for receiving an indicator substance, the chamber being connectable to the pocket via a passage area to be opened selectively,
b) bringing the applicator into contact with a sample substance,
c) introducing the applicator into the device, preferably into the pocket,
d) activating the device by exertion of pressure on at least one chamber or by pushing the applicator into a chamber, the applicator being brought into contact with an indicator substance,
e) optionally taking out the applicator after a period of time which is sufficient in order to generate a detectable signal,
f) optionally bringing the applicator into contact with a stop reagent in order to end the generation of the detectable signal after a period of time, the stop reagent being situated in a further chamber,
g) comparing the detectable signal with a reference signal,
h) optionally carrying out a positive control to check the functionality of the method for analysis.

2. The method as claimed in claim 1, after step c) the applicator firstly being brought into contact with a nutrient solution in a step c′).

3. The method as claimed in one of the above claims, the indicator substance being activated by mixing the indicator substance with an activating substance before activating the device as in step d).

4. The method as claimed in one of the above claims, the device being opened by at least partial removal or pulling-up of a film or of a film constituent before step a).

5. The method as claimed in one of the above claims, the sample substance being saliva and, following step g), an impression of the tissue which is rinsed by the saliva being produced using an impression compound, containing diagnostically active additives.

6. The use of a device, comprising a pocket for receiving an applicator and at least one chamber for receiving an indicator substance, the chamber being connectable to the pocket via a passage area to be opened selectively, for carrying out the method as claimed in one of claims 1 to 5.

7. The use of a device, comprising a pocket for receiving an applicator and at least one chamber for receiving an indicator substance, the chamber being connectable to the pocket via a passage area to be opened selectively, for carrying out an analysis, in particular of saliva, for the detection of caries-causing bacteria, and/or for the analysis of gum pocket fluid, in particular for the detection of parodontitis-causing bacteria.

8. A device, comprising a pocket, an applicator and at least one chamber, containing an indicator substance chosen from pH indicators, redox indicators, fluorescence indicators, chemoluminescence indicators, vitality indicators and color indicators, and optionally a further chamber, containing buffer substances, acids, bases, solvents, salts, enzymes, coenzymes, vitamins and/or stop reagents, the first chamber being connectable to the pocket via a passage area to be opened selectively and the optionally present second chamber being connectable to the first chamber and/or to the pocket via a passage area to be opened selectively.

9. The device as claimed in claim 8 having two or three chambers.

10. The device as claimed in one of claims 8 or 9, all chambers present being connected via passage areas to be opened selectively in such a way that all substances contained therein can be transferred directed serially and/or in parallel to the pocket.

11. The device as claimed in one of claims 8 to 10, the applicator having a self-tapping thread.

12. The device as claimed in one of claims 8 to 10, a reference signal, preferably a color scale, being attached to the device.

13. A kit for carrying out a method for analysis on caries- and/or parodontitis-causing bacteria, comprising a device as claimed in one of claims 8 to 12 and an impression compound, containing diagnostically active additives.

14. A kit for carrying out a method for analysis on caries- and/or parodontitis-causing bacteria, comprising a device as claimed in one of claims 8 to 12 and a container, comprising substances for carrying out a positive control for checking the functionality of the method for analysis.

15. The kit as claimed in claim 14, the container being connected to the device or being present as a separate constituent of the kit.
